(19) United States
(12) Patent Application Publication
(10) Pub. No.: US 2007/0255263 A1
(43) Pub. Date: Nov. 1, 2007

(54) LACRIMAL PASSAGE TREATMENT INSTRUMENT

(76) Inventor: Manabu Sugimoto, Souja-shi (JP)

Correspondence Address: SNELL & WILMER LLP (OC), 600 ANTON BOULEVARD, SUITE 1400, COSTA MESA, CA, 92626, US

(21) Appl. No.: 11/739,535

(22) Filed: Apr. 24, 2007

(30) Foreign Application Priority Data
Apr. 27, 2006 (JP) 2006-123510
Nov. 20, 2006 (JP) 2006-313629

(51) Int. Cl. A61F 2/04

(52) U.S. Cl. 606/1; 623/23.64

(57) ABSTRACT

A sheath as a lacrimal passage treatment instrument is provided. The sheath is composed of a flexible tube that is bendable and self-standable and has an outside diameter insertable into a lacrimal passage, and has both ends opened. One of the ends of the sheath has a decreasing wall thickness to form a taper, and slits are formed opposite to each other at the end.

### BACKGROUND OF THE INVENTION

1. Field of the Invention

The present invention relates to a lacrimal passage treatment instrument used for treating lacrimal passage obstructions and the like.

2. Prior Art

When a lacrimal passage becomes obstructed, firstly a bougie (a probe) alone is inserted into the lacrimal passage to dislodge an obstruction. Next, a tube is inserted into the lacrimal passage using a stylet, and then the stylet is removed to leave the tube in the lacrimal passage for eight weeks. After this, the tube is removed.

As the tube used for such a lacrimal passage treatment, two tubes (which constitute one whole tube) are inserted into the lacrimal passage as described, for example, in Japanese Patent Application Publication No. H07-213551.

To treat an obstructed lacrimal passage, a doctor firstly inserts a bougie into the lacrimal passage to push an obstruction in the lacrimal passage, thereby opening the lacrimal passage. During this operation, the bougie may break into a wall surface of the lacrimal passage, move forward, and then return into the lacrimal passage. This causes a false passage to be formed. After this, the doctor removes the boogie, and inserts a tube into the lacrimal passage using a stylet (a slender metal rod of stainless steel or the like, which is inserted in a flexible tube to maintain a shape of the tube). At this time, the doctor may erroneously insert the tube into the false passage. In such a case, after the stylet is removed, the tube remains inserted in the false passage.

This is just one example of a failure of correctly inserting a tube into a lacrimal passage. Since various states emerge in a treatment process, such a failure can frequently occur. If any one of the two tubes is inserted in the false passage, the lacrimal passage cannot be treated properly.

The present invention was conceived to solve the above problem, and aims to provide a lacrimal passage treatment instrument for assisting in a treatment to correctly insert a tube into a lacrimal passage.

### SUMMARY OF THE INVENTION

The stated aim can be achieved by a sheath used as a lacrimal passage treatment instrument, characterized by: being composed of a flexible tube that is bendable and self-standable, and has an outside diameter insertable into a lacrimal passage; and having both ends opened.

Here, one of the ends may have a decreasing wall thickness to form a taper.

Here, slits may be formed opposite to each other at the end.

Here, the flexible tube may be made of a transparent material or a semi-transparent material, with a mark for recognizing the slits being made at the end.

Here, a grip may be formed at one of the ends.

The stated aim can also be achieved by a lacrimal passage treatment instrument including: a body portion made of a flexible material and having an outside diameter insertable into a lacrimal passage; and a flexible guide portion formed at an end of the body portion and having a smaller outside diameter than the body portion.

Here, the guide portion and the body portion may be integrally formed.

Here, a projection stopper may be formed around one of the ends.

The above construction provides the specially designed sheath. By inserting a probe of a medical camera into this sheath, an obstruction in the lacrimal passage can be dislodged while observing the inside of the lacrimal passage, so that a tube can be prevented from being inserted in a false passage. In addition, the tube can be placed in the lacrimal passage speedily by using a guidewire.

Also, the provision of the guide portion on the body portion makes it unnecessary to use a guidewire, so that the body portion can be placed in the lacrimal passage more speedily. Furthermore, by forming the projection stopper around the end of the sheath, the sheath can be kept from completely sliding into the lacrimal passage from a lacrimal punctum even if the sheath is of a short length.

### BRIEF DESCRIPTION OF THE DRAWINGS

These and other objects, advantages and features of the invention will become apparent from the following description thereof taken in conjunction with the accompanying drawings which illustrate a specific embodiment of the invention.

In the drawings:

FIG. 1 is a plan view of part of a lacrimal passage treatment instrument to which the present invention relates;

FIG. 2 is a sectional view of a lacrimal passage treatment instrument according to one example of the present invention;

FIG. 3 is a perspective view of a lacrimal passage treatment instrument according to another example of the present invention;

FIGS. 4A and 4B are respectively a side view and a plan view of part of a camera used for a lacrimal passage treatment;

FIG. 5 is a sectional plan view of a lacrimal passage showing a state in a treatment;

FIG. 6 is a sectional plan view of the lacrimal passage showing another state in the treatment;

FIG. 7 is a sectional plan view of the lacrimal passage showing another state in the treatment;

FIG. 8 is a sectional plan view of the lacrimal passage showing another state in the treatment;

FIG. 9 is a sectional plan view of the lacrimal passage showing another state in the treatment;

FIG. 10 is a sectional plan view of the lacrimal passage showing another state in the treatment;

FIG. 11 is a sectional plan view of the lacrimal passage showing a state in another treatment;

FIG. 12 is a sectional plan view of the lacrimal passage showing a state in another treatment;

FIG. 13 is a sectional plan view of the lacrimal passage showing a state in another treatment;

FIG. 14 is a plan view of a tube according to another example of the present invention;

FIG. 15 is a sectional view of a lacrimal passage treatment instrument according to another example of the present invention;

FIG. 16 is a perspective view of the lacrimal passage treatment instrument shown in FIG. 15;

FIG. 17 is a sectional plan view of the lacrimal passage showing a state in a treatment that uses the lacrimal passage treatment instrument shown in FIG. 15;

FIG. 18 is a sectional plan view of the lacrimal passage showing another state in the treatment that uses the lacrimal passage treatment instrument shown in FIG. 15; and

FIG. 19 is a sectional plan view of the lacrimal passage showing another state in the treatment that uses the lacrimal passage treatment instrument shown in FIG. 15.

### DESCRIPTION OF THE PREFERRED EMBODIMENT(S)

The following describes an embodiment of the present invention with reference to drawings.

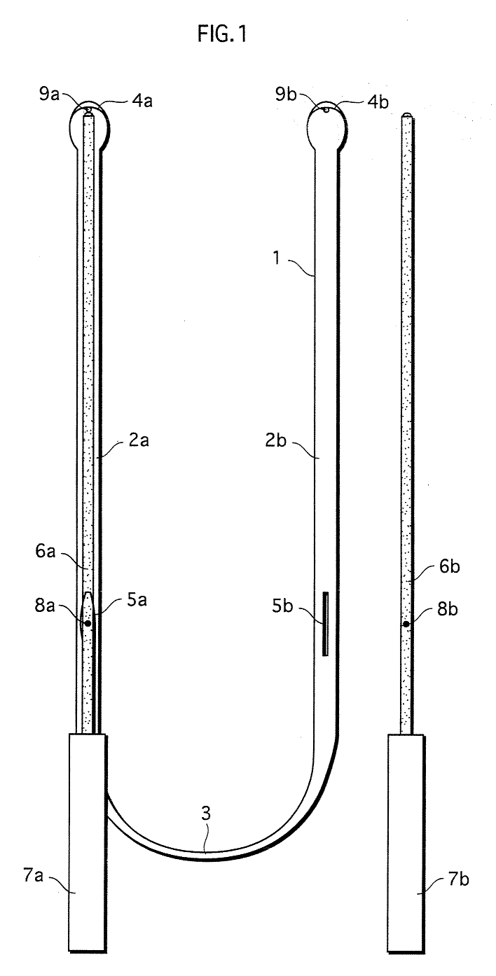

FIG. 1 is a plan view of an example of a lacrimal passage intubation instrument that may constitute part of a lacrimal passage treatment instrument to which the present invention relates.

In FIG. 1, reference numeral 1 denotes a flexible, transparent tube made of a resin such as silicon or polyurethane. The tube 1 is composed of tubes 2a and 2b and a thin, solid small-diameter portion 3 that connects the tubes 2a and 2b in the middle. Ends of the tubes 2a and 2b away from the small-diameter portion 3 are curved outward in a shape of a sphere, an oval sphere, or the like, thereby forming swollen tips 4a and 4b whose walls are made slightly thicker. The other ends of the tubes 2a and 2b near the small-diameter portion 3 respectively have slits (openings) 5a and 5b which are insertion slots. As one example, the tubes 2a and 2b have an outside diameter of 1.0 mm, an inside diameter of 0.5 mm, and a length of 90 mm in the case of a short-type tube and 105 mm in the case of a standard-type tube.

One pair of stylets (probes) 6a and 6b are each composed of a metal rod of stainless steel or the like, and have handles 7a and 7b at their ends. As one example, the stylets 6a and 6b have a diameter of 0.45 mm, and a length of 55 mm excluding the handles 7a and 7b.

The stylet 6a is inserted into the slit 5a from its tip, until the tip of the stylet 6a is located near the tip 4a of the tube 2a. In this state, a mark 8a is made on the stylet 6a at a position corresponding to the slit 5a, to keep the tip of the stylet 6a within the tube 2a (i.e. to prevent the tip of the stylet 6a from breaking through the tip 4a of the tube 2a). The tip of the stylet 6a is set at a predetermined position by visually observing this mark 8a. The same applies to the relation between the stylet 6b and the tube 2b, with a mark 8b being made on the stylet 6b.

Holes 9a and 9b are formed respectively at the tips 4a and 4b of the tubes 2a and 2b, by laser processing or the like. These holes 9a and 9b are used to pass a guide through (described later).

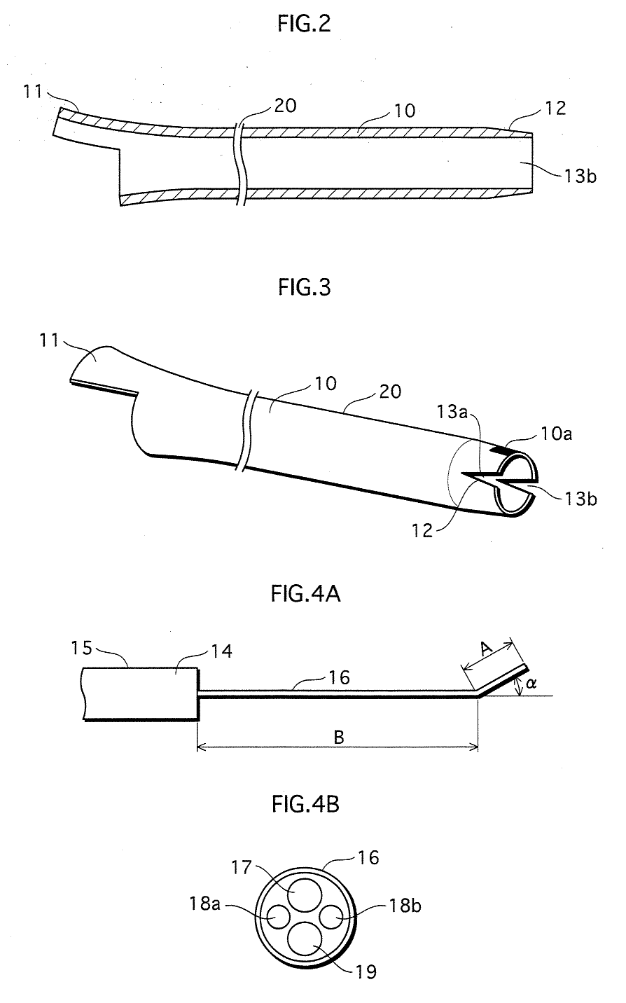

FIG. 2 is a sectional view of a lacrimal passage treatment sheath 20 that is a characteristic part of the present invention. A sheath body 10 of the sheath 20 is composed of a transparent or semi-transparent cylinder (tube) made of a flexible material such as silicon, polyurethane, polyethylene, Teflon (registered trademark), or the like. The sheath body 10 has the following degree of flexibility. The sheath body 10 is not as soft as the tubes 2a and 2b shown in FIG. 1. Rather, the sheath body 10 can maintain a standing position when erected vertical by holding its lower end (self-standable) Also, the sheath body 10 is bent when pressed by holding both ends, but returns to the original straight state when the hold is released.

The sheath body 10 which is a cylinder have both ends opened. A grip 11 is integrally formed at one end of the sheath body 10. This grip 11 is formed by removing part of the sheath body 10 from the end as illustrated. The grip 11 may have a length of about 2 mm. The grip 11 is slightly bent outward, as shown in FIG. 2. Also, the end of the sheath body 10 to which the grip 11 is attached is entirely made larger in diameter. This can be done, for example, by pressing a heated metal conical body into the sheath body 10. Meanwhile, the other end of the sheath body 10 is processed so as to become thinner toward a tip, thereby forming a taper 12. As one example, the sheath body 10 has an outside diameter of 1.1 mm, a wall thickness of 0.1 mm, and a length of 4.5 cm (which is longer than the distance from a lacrimal punctum to a lower end of a nasolacrimal duct).

Though the sheath 20 shown in FIG. 2 has only the taper 12 at the end, slits 13a and 13b may be formed opposite to each other at the end of the sheath 20 for a special purpose, as shown in FIG. 3. A function of these slits 13a and 13b will be described in detail later. A mark 10a for recognizing the slits 13a and 13b is made at the extreme edge of the flexible cylinder which is made of the transparent or semi-transparent material.

Parts other than the slits 13a and 13b in FIG. 3 are the same as those in FIG. 2.

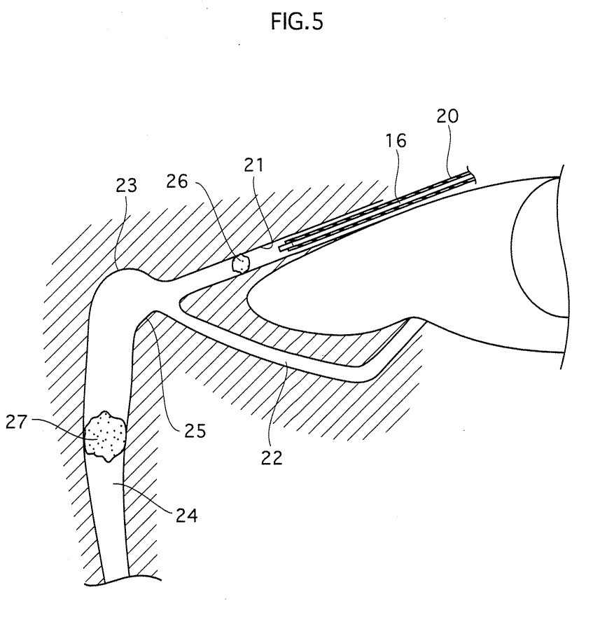

FIGS. 4a and 4b show a medical camera 14. The medical camera 14 is composed of a body part 15 and a probe (an exploration rod) 16 that projects from the body part 15. This medical camera 14 has already been used by ophthalmologists. An end of the probe 16 is bent by an angle α of about 27 degrees. A length A of this bent portion of the probe 16 is about 10 mm, whereas a length B of the linear portion of the probe 16 is about 4 cm.

A conduit 17 for conveying water, optical fibers 18a and 18b which serve as a light source, and a lens 19 are provided inside the probe 16, as shown in FIG. 4B.

In addition to the sheath 20 and the medical camera 14, a guidewire and, according to need, a bougie are necessary for conducting a treatment. The guidewire is preferably a metal wire of silver, copper, or the like with a diameter of about 0.1 mm. Alternatively, a resin wire may be used so long as it is strong and elastic and has a diameter easily insertable into the sheath body 10.

The bougie has already been mentioned in the above description of the prior art, and is composed of an elastic metal rod of stainless steel or the like with a diameter of about 0.4 mm to 0.6 mm.

The following describes a method of treating a lacrimal passage by using the sheath 20, the medical camera 14, the guidewire, and, according to need, the bougie.

Firstly, the sheath 20 is put over the probe 16 of the medical camera 14 from the end having the grip 11, until the end of the probe 16 extends from the other end of the sheath body 10 by about 0.5 mm.

FIG. 5 shows a state of the lacrimal passage. Reference numeral 21 denotes a superior lacrimal canaliculus, reference numeral 22 denotes an inferior lacrimal canaliculus, reference numeral 23 denotes a lacrimal sac, reference numeral 24 denotes a nasolacrimal duct, reference numeral 25 denotes an elevation, reference numeral 26 denotes an obstruction lodged in the lacrimal canaliculus 21, and reference numeral 27 denotes an obstruction lodged in the nasolacrimal duct 24.

The probe 16 of the medical camera 14 set in the above state is introduced into the lacrimal canaliculus 21 from the sheath 20 side, as shown in FIG. 5. While observing the state of obstruction in the lacrimal canaliculus 21 by the medical camera 14, the probe 16 is moved farther into the lacrimal canaliculus 21. When the obstruction 26 is found, the obstruction 26 is dislodged by pushing with the end of the sheath 20 and the end of the probe 16.

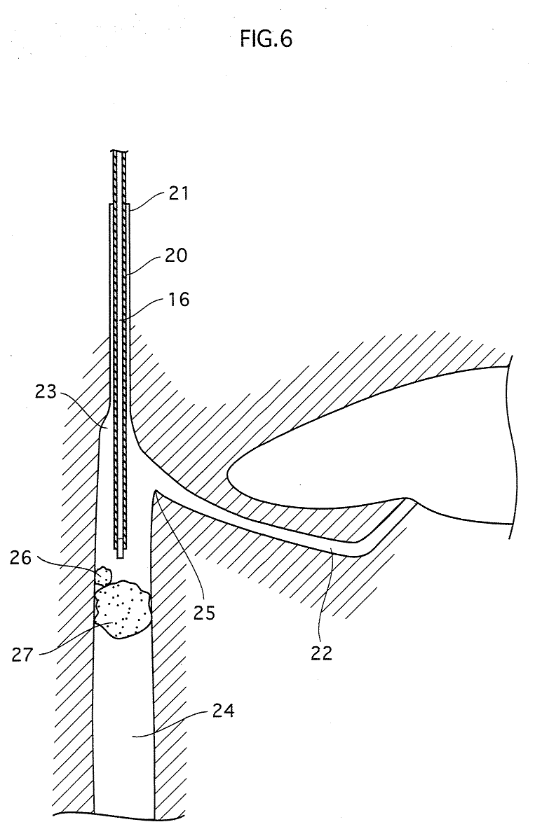

After it is confirmed that the end of the probe 16 has entered the lacrimal sac 23, the probe 16 is erected as shown in FIG. 6. From this state, the probe 16 is moved farther into the nasolacrimal duct 24, while observing the state of obstruction in the nasolacrimal duct 24 by the medical camera 14. When the obstruction 27 is found, the obstruction 27 is dislodged by pushing with the end of the sheath 20 and the end of the probe 16.

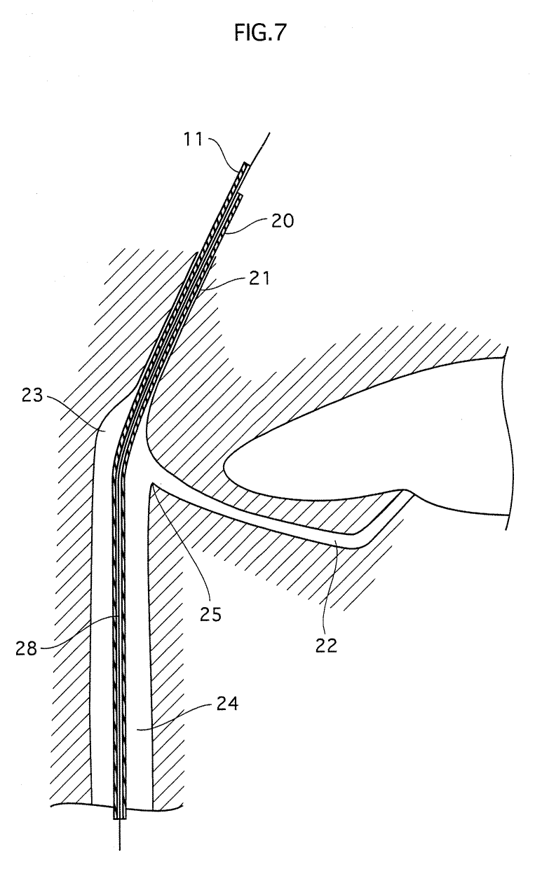

Next, in a state where the end of the sheath 20 extends from a lower end of the nasolacrimal duct 24, the probe 16 of the medical camera 14 is removed from the lacrimal punctum side while fixing the sheath 20 at the grip 11. As a result, only the sheath 20 is left in the lacrimal passage. In this state, a guidewire 28 mentioned above is passed through the sheath 20 as shown in FIG. 7. Following this, the sheath 20 is removed from the lacrimal passage by holding the grip 11 of the sheath 20 with tweezers. With the provision of the grip 11, the sheath 20 can be easily removed from the lacrimal passage.

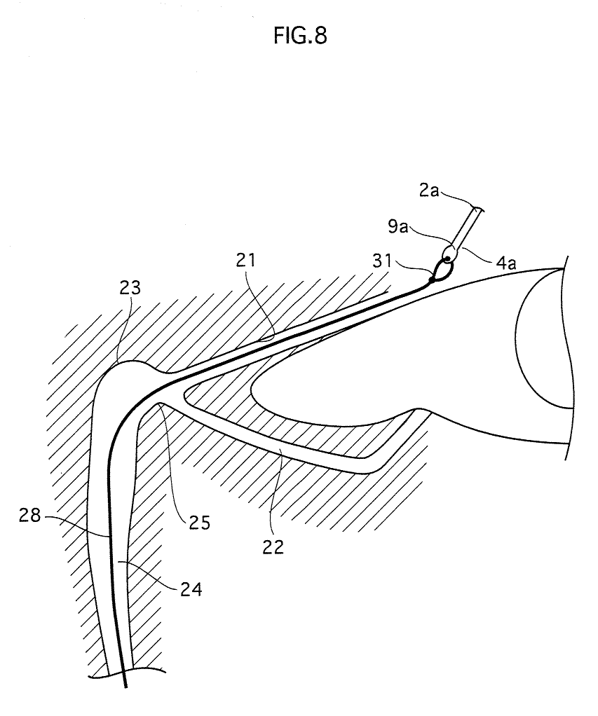

This leaves only the guidewire 28 in the lacrimal passage. As shown in FIG. 8, an end of the guidewire 28 is passed through the hole 9a of the tube 2a, and the guidewire 28 is firmly connected to the tube 2a by making the end of the guidewire 28 into a ring and bonding with an adhesive and the like.

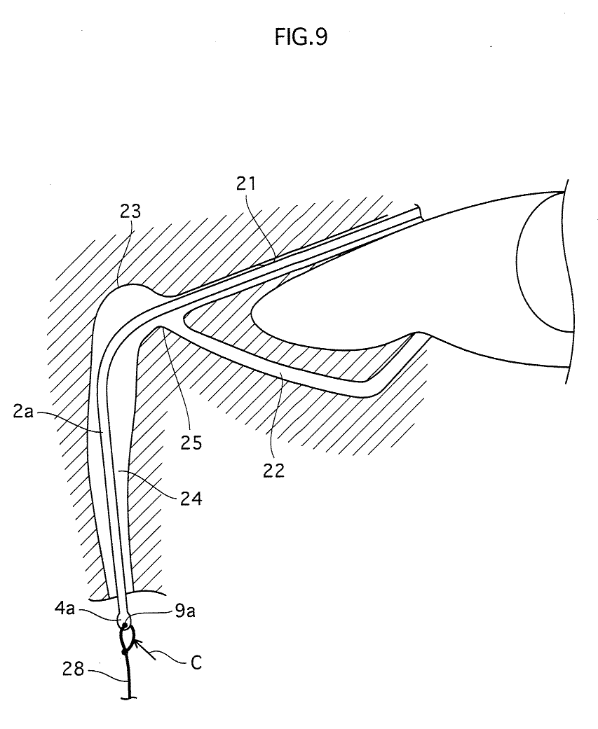

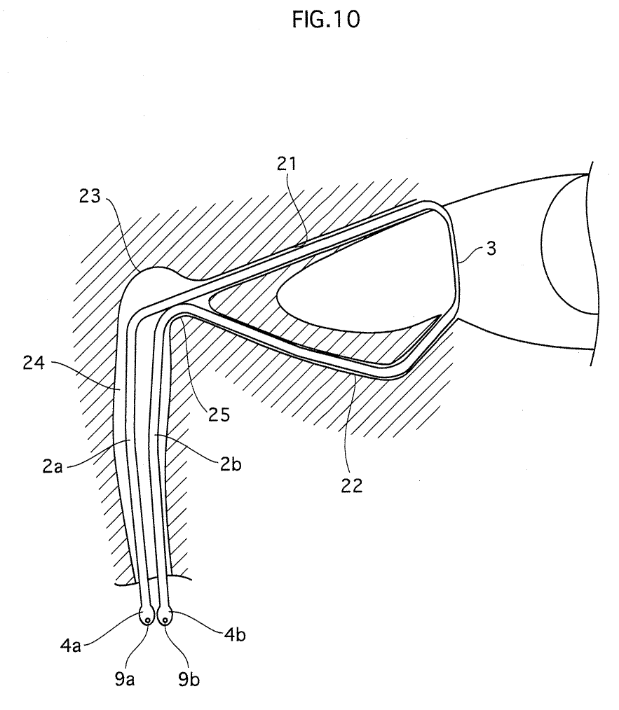

Next, a lower end of the guidewire 28 is pulled by holding with tweezers. As a result, the guidewire 28 comes out of the lacrimal passage and the tube 2a enters the lacrimal passage, as shown in FIG. 9. The guidewire 28 is cut at a position indicated by the arrow C in FIG. 9, to disconnect the guidewire 28 from the tube 2a.

In the same manner, the tube 2b is inserted into the inferior lacrimal canaliculus 22 and the nasolacrimal duct 24. Thus, the tubes 2a and 2b can be placed in the lacrimal passage.

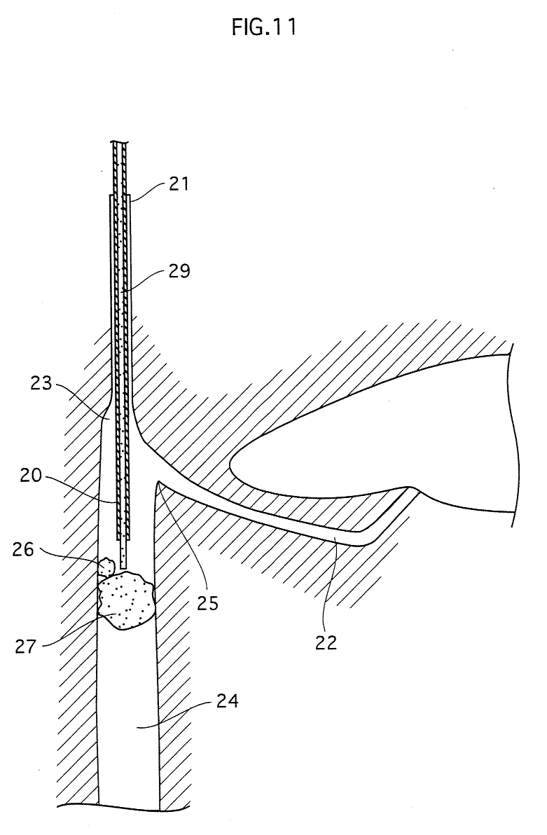

In FIG. 6, the obstruction 27 is dislodged using the sheath 20 and the probe 16. This can work when the obstruction 27 is soft. However, if the obstruction 27 is hard, it may be unable to be dislodged using the sheath 20 and the probe 16. In such a case, as shown in FIG. 11, the probe 16 is removed from the sheath 20, and instead a bougie 29 is inserted into the sheath 20 to dislodge the obstruction 27. Since the bougie 29 is slender and hard, even a hard obstruction can be easily dislodged by using the bougie 29.

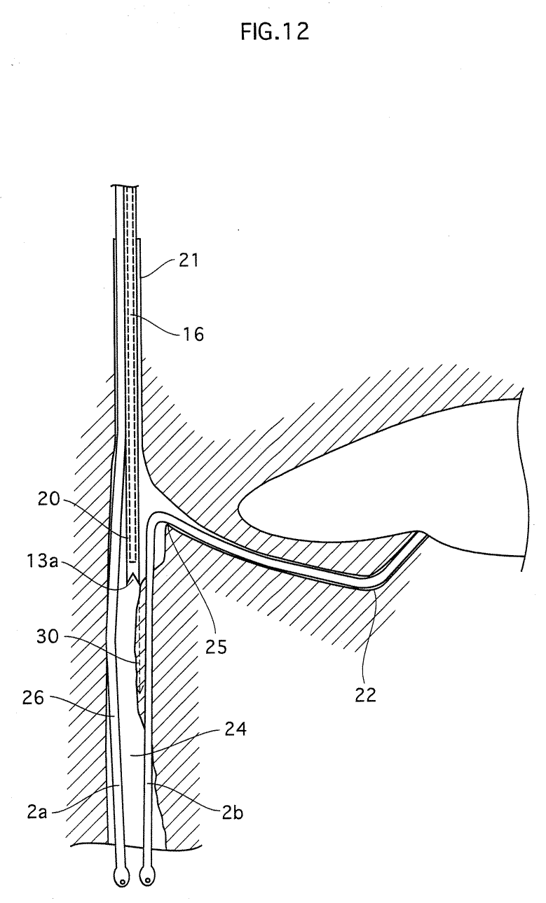

Also, there may be cases where a treatment of placing the tubes 2a and 2b in the lacrimal passage by using the stylets 6a and 6b shown in FIG. 1 and a bougie is considered appropriate. In such a treatment, however, the tube 2b will often end up being placed in a false passage (a passage formed as a result of breaking into the wall of the lacrimal passage) while the tube 2a can successfully be placed in the lacrimal passage, as shown in FIG. 12.

In this case, the tube 2b can be placed in the lacrimal passage, by cutting a mucous membrane 30 jutting into the nasolacrimal duct 24 using the shovel-shaped end of the sheath 20 formed by the slits 13a and 13b and the taper 12. When doing so, by monitoring the mark 10a by the medical camera 14 through the probe 16, it is possible to check whether the slits 13a and 13b are positioned properly, that is, whether the shovel portion of the sheath 20 is in contact with the mucous membrane 30.

In the above way, the obstructions 26 and 27 in the lacrimal passage can be dislodged and the tubes 2a and 2b can be placed in the lacrimal passage. The tubes 2a and 2b are removed from the lacrimal passage after about eight weeks, to complete the treatment.

In the above lacrimal passage treatment, a knot 31 of the guidewire 28 in FIG. 8 needs to be tied firmly. When pulling the guidewire 28 from the lower end of the nasolacrimal duct 24 to have the tube 2a enter the lacrimal passage, if the knot 31 becomes untied, the tube 2a cannot be placed in the lacrimal passage. Also, if the knot 31 is too large, the knot 31 may get caught in the lacrimal passage, making it impossible to pass the guidewire 28 through. Besides, the knot 31 can damage the wall surface of the lacrimal passage. Furthermore, since the guidewire 28 comes in direct contact with the wall surface of the lacrimal passage especially at a bent part between the lacrimal canaliculus 21 and the lacrimal sac 23, this part may get damaged by the guidewire 28.

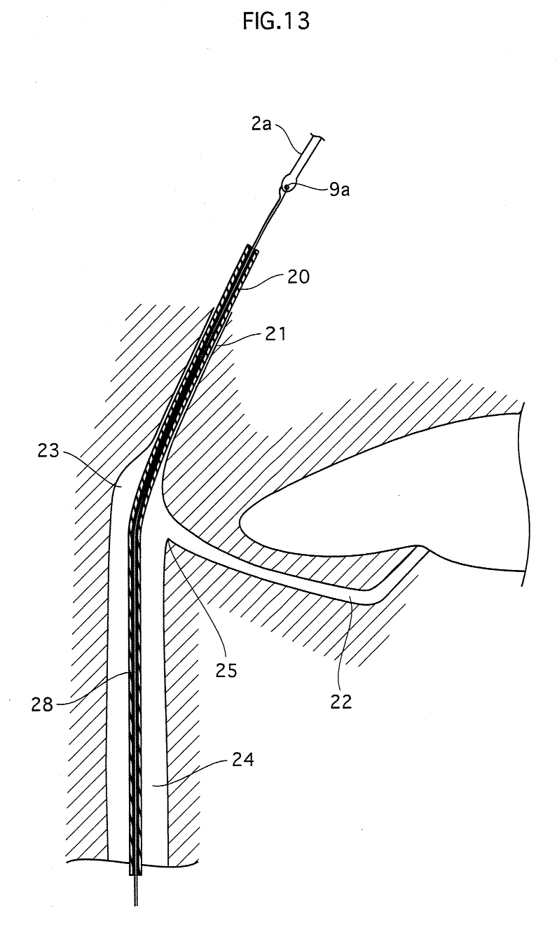

In view of this, the sheath 20 from which the grip 11 is removed may be used as shown in FIG. 13 (the grip 11 may be cut later). Using this sheath 20, the obstructions 26 and 27 are dislodged and then the probe 16 is removed, as described earlier. After this, the guidewire 28 is passed through the hole 9a of the tube 2a as shown in FIG. 13. Here, the guidewire 28 is not tied as shown in FIG. 8. Instead, the guidewire 28 is folded in half to form two wires, which are inserted into the sheath 20 and pulled out from the lower end of the nasolacrimal duct 24. When the guidewire 28 is pulled, the tip of the tube 2a contacts against the end of the sheath 20. Since the sheath 20 flares out at the end, the tip of the tube 2a enters into the end of the sheath 20, with it being possible to favorably remove the sheath 20 from the lacrimal passage.

In a state where the end of the guidewire 28 and the end of the sheath 20 are exposed, by pulling the sheath 20, the tip of the tube 2a is brought into contact against the end of the sheath 20. As the guidewire 28 and the sheath 20 are pulled, the sheath 20 comes out of the lacrimal passage while the tube 2a enters the lacrimal passage. Lastly, by removing the guidewire 28 from the hole 9a of the tube 2a, the tube 2a can be placed in the lacrimal passage. According to this method, the inner wall of the lacrimal passage can be protected from damage by the guidewire 28.

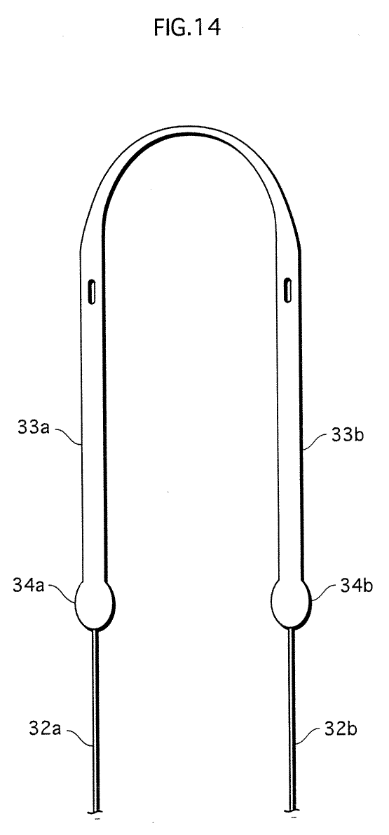

Also, a lacrimal passage treatment instrument shown in FIG. 14 may be used. This lacrimal passage treatment instrument includes body portions 33a and 33b (corresponding to the tubes 2a and 2b in FIG. 1) and small-diameter (about 0.3 mm to 0.5 mm) guide portions 32a and 32b (corresponding to the guidewire 28) which are integrally formed with the body portions 33a and 33b at their ends 34a and 34b (corresponding to the tips 4a and 4b in FIG. 1). A length of each of the guide portions 32a and 32b is set such that the guide portion extends from the opening of the nasolacrimal duct by about 5 cm.

The body portions 33a and 33b and the guide portions 32a and 32b are made of a material such as silicon, polyurethane, or the like, and have sufficient flexibility. Though the body portions 33a and 33b are tubular in FIG. 14, they may instead be solid. Also, the body portions 33a and 33b and the guide portions 32a and 32b may be separately formed and connected by an adhesive.

When using the lacrimal passage treatment instrument shown in FIG. 14, the guidewire 28 need not be used. The guide portion 32a is inserted from the end of the sheath 20 inserted in the lacrimal passage, and pulled from the lower end of the lacrimal passage. When the guide portion 32a is pulled, the sheath 20 comes out of the lower end of the lacrimal passage, and the body portion 33a enters the lacrimal passage. Thus, the sheath 20 is removed by holding the guide portion 32a and the sheath 20. Lastly, the guide portion 32a is cut from the body portion 33a. In this way, the body portion 33a can be placed in the lacrimal passage. The same applies to the body portion 33b.

According to this method, there is no need to use a guidewire, so that the treatment can be performed more speedily.

If the state of the lacrimal passage makes it impossible to use the sheath 20 and the medical camera 14, the guide portion 32a shown in FIG. 14 may be cut from its base, so that a general treatment is performed on the lacrimal passage using a stylet while the other lacrimal passage is treated using the sheath 20 and the guide portion 32b. This contributes to convenience.

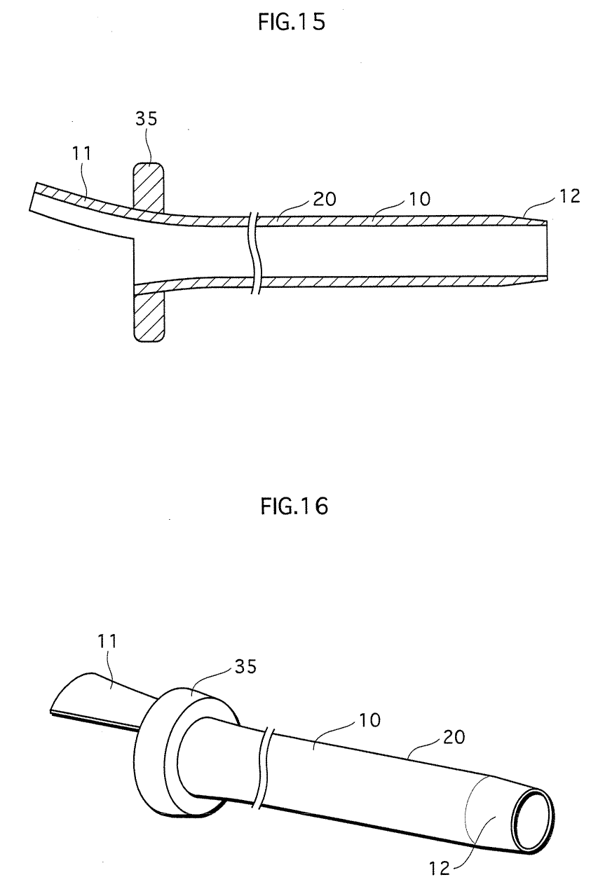

FIGS. 15 and 16 are respectively a sectional view and a perspective view of a sheath of another example. In the sheath 20 of this example, the sheath body 10 has a short length of about 20 mm. A ring-shaped stopper 35 is provided at the end of this sheath 20.

For example, a perforated bead made of a resin may be used for this stopper 35. In detail, an adhesive is applied to the sheath body 10, and the bead is engaged with the sheath body 10. The bead has a diameter of about 2 mm to 3 mm.

Here, the stopper 35 is not limited to the ring shape. For instance, two or three projection pieces may be provided around the end of the sheath body 10. In other words, the stopper 35 can be realized by any projection stopper such as a ring, a projection piece, and the like.

During the treatment where the sheath 20 is inserted in the lacrimal passage from the lacrimal punctum, when the sheath 20 is short, the sheath 20 may be completely inserted in the lacrimal passage. If this happens, it is difficult to remove the sheath 20 from the lacrimal passage. In view of this, the stopper 35 is provided to prevent the sheath 20 from completely entering into the lacrimal passage.

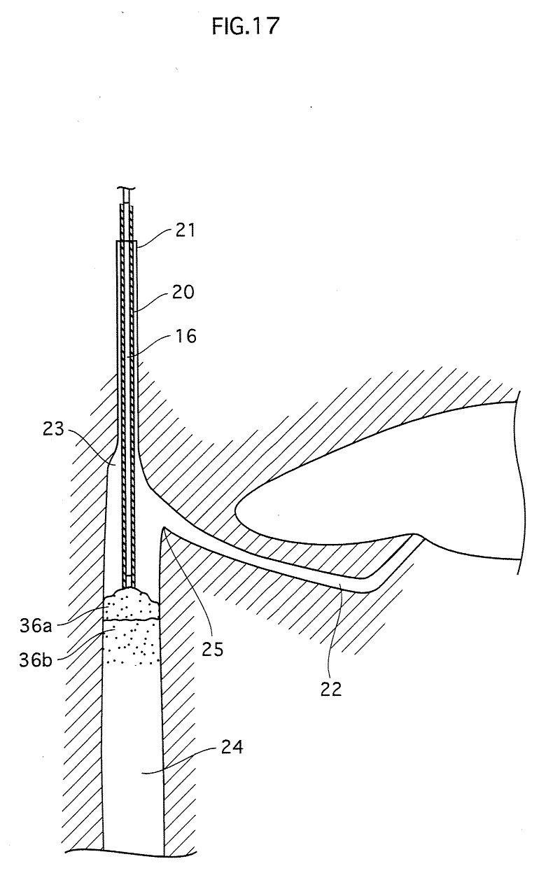

The following describes a treatment method that uses this short sheath 20. As shown in FIG. 17, the sheath 20, in which the probe 16 of the medical camera 14 is inserted, is introduced into the superior lacrimal canaliculus 21, and the end of the sheath 20 is brought into contact with an obstruction 36a lodged in the lacrimal passage. If the obstruction 36a is soft, it can be dislodged by pushing with the end of the probe 16 and the end of the sheath 20. If the obstruction 36a is hard, however, it cannot be dislodged even if pushed with the end of the probe 16 and the end of the sheath 20.

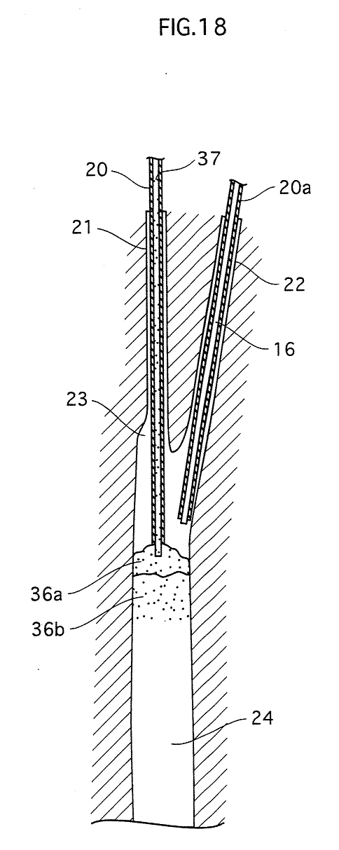

In such a case, the probe 16 is removed from the sheath 20 in the above state. Next, another sheath 20a, in which the probe 16 is inserted, is introduced into the inferior lacrimal canaliculus 22 as shown in FIG. 18. In this state, a bougie 37 is inserted into the sheath 20, and the obstruction 36a is dislodged using the bougie 37 while observing the obstruction 36a by the medical camera 14 through the probe 16. Reference numeral 36b denotes a soft obstruction which exists below the hard obstruction 36a.

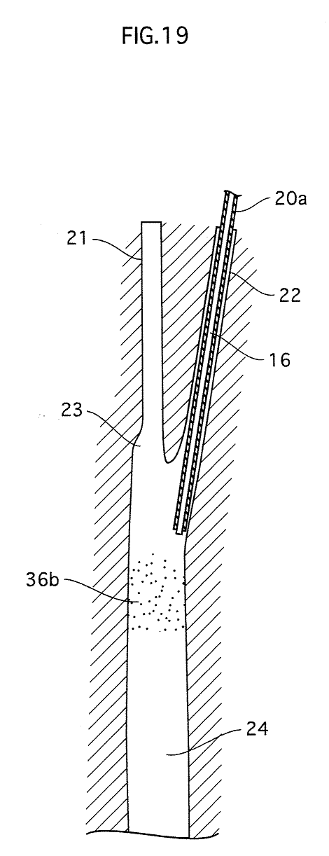

After this, the bougie 37 and the sheath 20 are removed, and the obstruction 36b is pushed down by the end of the probe 16 and the end of the sheath 20a, as shown in FIG. 19.

When performing such a treatment, the sheaths 20 and 20a can be of short length, but the short sheaths 20 and 20a may completely enter into the lacrimal passage. If the sheaths 20 and 20a are each provided with the stopper 35, they can be prevented from completely entering into the lacrimal passage.

The stopper 35 may also be provided in the long sheaths shown in FIGS. 2 and 3.

Although the present invention has been fully described by way of examples with reference to the accompanying drawings, it is to be noted that various changes and modifications will be apparent to those skilled in the art.

Therefore, unless such changes and modifications depart from the scope of the present invention, they should be construed as being included therein.

1. A sheath used as a lacrimal passage treatment instrument, characterized by:
being composed of a flexible tube that is bendable and self-standable, and has an outside diameter insertable into a lacrimal passage; and
having both ends opened.

2. The sheath of claim 1,
wherein one of the ends has a decreasing wall thickness to form a taper.

3. The sheath of claim 2,
wherein slits are formed opposite to each other at the end.

4. The sheath of claim 3,
wherein the flexible tube is made of a transparent material or a semi-transparent material, with a mark for recognizing the slits being made at the end.

5. The sheath of claim 1,
wherein a grip is formed at one of the ends.

6. A lacrimal passage treatment instrument comprising:
a body portion made of a flexible material and having an outside diameter insertable into a lacrimal passage; and
a flexible guide portion formed at an end of the body portion and having a smaller outside diameter than the body portion.

7. The lacrimal passage treatment instrument of claim 6,
wherein the guide portion and the body portion are integrally formed.

8. The sheath of claim 1,
wherein a projection stopper is formed around one of the ends.

9. The sheath of claim 5,
wherein a projection stopper is formed around the end.

10. The sheath of claim 8,
wherein the projection stopper has a ring shape.

11. The sheath of claim 9,
wherein the projection stopper has a ring shape.
